(19) United States
(12) Patent Application Publication
(10) Pub. No.: US 2016/0031846 A1
(43) Pub. Date: Feb. 4, 2016

(54) KETAL ADDUCTS, METHODS OF MANUFACTURE, AND USES THEREOF

(71) Applicant: SEGETIS, INC., Golden Valley, MN (US)

(72) Inventors: BRIAN D. MULLEN, DELANO, MN (US); DORIE J. YONTZ, BLOOMINGTON, MN (US); MARC D. SCHOLTEN, SAINT PAUL, MN (US)

(21) Appl. No.: 14/777,149

(22) PCT Filed: Mar. 12, 2014
(86) PCT No.: PCT/US2014/024216

§ 371 Date: Sep. 15, 2015

Related U.S. Application Data
(60) Provisional application No. 61/794,618, filed on Mar. 15, 2013.

(51) Int. Cl. C07D 317/30; A61K 9/107; A61Q 19/00; A61K 8/49; A61K 47/22

(52) U.S. Cl. CPC C07D 317/30; A61K 8/4973; A61K 47/22; A61Q 19/00; A61K 9/107

(57) ABSTRACT

A ketal amide has a structure represented by formula (I):

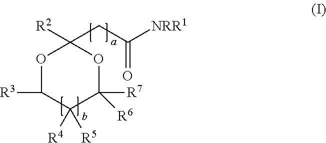

wherein R is hydrogen or C₁₋₈ alkyl; R¹ is substituted or unsubstituted, saturated or unsaturated C₁₋₃₆ alkyl, or an alkylene oxide of the formula (CₙH₂ₙO)ₚCₙH₂ₙOR^{a} wherein n is 1-4, p is 1-1000 and R^{a} is H or CₙH₂ₙ₊₁ wherein n is 1 to 4, R² is hydrogen or C₁₋₃ alkyl, each R³, R⁴, and R⁵ is independently hydrogen or C₁₋₆ alkyl, R⁶ is hydrogen or C₁₋₆ alkyl, R⁷ is C₁₋₆ alkyl substituted with 1-4 hydroxyl groups, a is 0-3, and b is 0-1. Methods to prepare the ketal amide and compositions containing the ketal amide are also disclosed.

### BACKGROUND

This application relates to ketal adducts, methods of manufacture and the use of ketal adducts as surfactants in various compositions.

Surfactants are compounds that lower the surface tension of a liquid, the interfacial tension between two liquids, or that between a liquid and a solid. Surfactants may act as detergents, wetting agents, emulsifiers, foaming agents, and/or dispersants.

### SUMMARY

A variety of ketal esters and amides are disclosed for use as surfactants. The surfactants can be used as detergents, wetting agents, emulsifiers, foaming agents, and/or dispersants.

Also disclosed is an emulsion or microemulsion comprising a dispersed liquid phase, a continuous liquid phase, and the above-described ketal surfactants, which can be in either phase. In one embodiment, the ketal surfactants are in the continuous liquid phase. In another embodiment, the ketal surfactants are in the dispersed liquid phase. In yet another embodiment, the ketal surfactants are partially in both phases including at the interface.

A method for the manufacture of the emulsion comprises dispersing a first liquid into a second liquid in the presence of the above-described ketal surfactant to form the emulsion.

Compositions comprising the emulsions are also disclosed, including a personal care composition, a drug delivery composition, an agricultural composition, a fragrance composition, a biocide composition, including pesticides, herbicides and fungicides and a cleaning composition.

A composition comprising the ketal surfactant can comprise at least one of the ketal surfactants and a solvent, such as water or an organic solvent. The compositions can be solutions, emulsions or microemulsions. The compositions can also contain additional components, such as pigments and resins. The compositions can be made into formulations which can be sprayed, poured, spread, coated, dipped or rolled.

The above described and other embodiments are further described in the drawings and detailed description that follow.

### BRIEF DESCRIPTION OF THE DRAWINGS

The following figures are representative embodiments, wherein the like elements are numbered alike.

FIG. 1 is a graph showing the creaming height of sesame oil/water emulsions as a function of freeze-thaw cycles stabilized with 3-dioxolane-2-propanoic acid, 4-(hydroxymethyl)-2-methyl-, lauramide (DPHML, or LGK lauramide) and cocoamide DEA.

FIG. 2 is a graph showing the time dependence of creaming height of sesame oil/water emulsions stabilized with DPHML (LGK lauramide) and cocoamide DEA at 35° C.

FIG. 3 is a graph showing the time dependence of creaming height of sesame oil/water emulsions stabilized with DPHML (LGK lauramide) and cocoamide DEA at 20° C.

FIG. 4 is a graph showing the time dependence of creaming height of sesame oil/water emulsions stabilized with DPHML (LGK lauramide) and cocoamide DEA at 5° C.

### DETAILED DESCRIPTION

A large number of chemical compounds and compositions are available as surfactants. Nonetheless, for many applications, there is an ongoing need for further improvements. Further, there is an increasing desire for “bio-sourced” emulsifiers that can be used as replacements for petroleum-sourced emulsifiers. It would be a further advantage if such emulsifiers were acceptable for use in cleaning and personal care applications, such as detergents and cosmetics.

The inventors hereof have discovered that certain ketal adducts such as ketal levulinic amides, are excellent surfactants. In some embodiments, the ketal levulinic amides are superior stabilizers of oil/water emulsions over currently used emulsifiers. This has been demonstrated for a variety of temperatures and over multiple freeze-thaw cycles. The ketal surfactants are of general applicability for commercial products, offering better shelf stability and ultimately extending the lifetime of said products. In a particularly advantageous feature, the ketal surfactants are bio-sourced, rather than petroleum-derived.

The ketal surfactants referred to herein can have the formula (I):

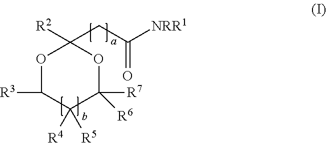

wherein

R is hydrogen or C₁₋₈ alkyl,

R¹ is substituted or unsubstituted, saturated or unsaturated C₁₋₃₆ alkyl, or an alkylene oxide of the formula (CₙH₂ₙO)ₚCₙH₂ₙOR^{a} wherein n is 1-3, p is 1-1000, 2-500, or 2-100, or 2-50, or 2-30, and R^{a} is H or CₙH₂ₙ₊₁ wherein n is 1 to 3,

R² is hydrogen or C1-3 alkyl,

each R³, R⁴, and R⁵ is independently hydrogen or C₁₋₆ alkyl,

R⁶ is hydrogen or C1-6 alkyl,

R⁷ is C₁₋₆ alkyl substituted with 1-4 hydroxyl groups,

a is 0-3, and

b is 0-1.

More specifically, R is hydrogen, R¹ is substituted or unsubstituted, saturated or unsaturated C₈₋₃₆ alkyl, polypropylene oxide, or polyethylene oxide, R² is methyl, each R³, R⁴, and R⁵ is independently hydrogen or C₁₋₃ alkyl, R⁶ is hydrogen, R⁷ is C₁₋₆ alkyl substituted with 1-4 hydroxyl groups, a is 1-3, and b is 0-1.

Even more specifically R is hydrogen, R¹ is substituted or unsubstituted, saturated or unsaturated C₈₋₃₆ alkyl, or polyethylene oxide, R² is methyl, R³ is hydrogen, R⁶ is hydrogen, R⁷ is C₁₋₄ alkyl substituted with 1-4 hydroxyl groups, a is 2-3, and b is 0 or 1. In an embodiment, b is 0.

In any of the foregoing embodiments, R¹ can be an unsubstituted or substituted, saturated or unsaturated C₈₋₂₈ alkyl, C₁₀₋₂₄ alkyl, C₁₀₋₂₀ alkyl, or C₁₂₋₁₈ alkyl; or R¹ can be an unsubstituted, saturated or unsaturated C₈₋₂₈ alkyl, C₁₀₋₂₄ alkyl, C₁₀₋₂₀ alkyl, or C₁₂₋₁₈ alkyl; or R¹ can be an unsubstituted, saturated C₈₋₂₈ alkyl, C₁₀₋₂₄ alkyl, C₁₀₋₂₀ alkyl, or C₁₂₋₁₈ alkyl. In an embodiment, R¹ is an unsubstituted, saturated C₁₂ alkyl or an unsubstituted, saturated C₁₈ alkyl. In another embodiment, R¹ is a polypropylene oxide (PPO), polyethylene oxide (PEO), or a mixed PPO-PEO wherein the propylene oxide and ethylene oxide units are present randomly or in alternating blocks.

In a specific embodiment, the ketal amide is of the formula (Ia):

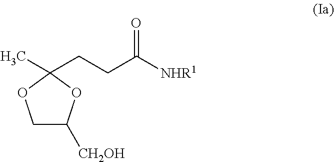

wherein R¹ is a C₈₋₃₆ alkyl, specifically a C₁₂₋₁₇ alkyl, PPO, PEO, or mixed PPO-PEO. Ketal amide (Ia) is an amide of the glyceryl ketal of levulinic acid (LGK). LGK stearamide is obtained when R¹ is a stearyl (C₁₈) group in formula (Ia), and LGK lauramide (DPHML) is obtained when R¹ is a lauryl group (C₁₂) in formula (Ia).

In another specific embodiment, the ketal amide is of the formula (Ib):

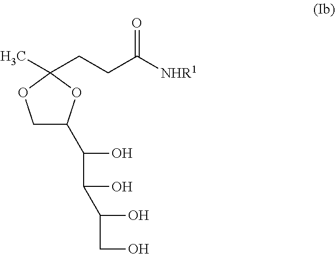

wherein R¹ is a C₈₋₃₆ alkyl, specifically a C₁₂₋₁₇ alkyl, PPO, PEO, or mixed PPO-PEO.

The ketal adduct can be a ketocarboxy ester of the formula (II):

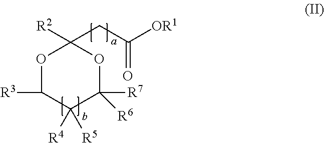

wherein

R¹ is substituted or unsubstituted, saturated or unsaturated C₈₋₃₆ alkyl or an alkylene oxide of the formula (CₙH₂ₙO)CₙH₂ₙOR^{a} wherein n is 1-3, m is 1-40, 1 to 30, or 2-20 and R^{a} is H or CₙH₂ₙ₊₁ wherein n is 1 to 3,

R² is hydrogen or C1-3 alkyl,

each R³, R⁴, and R⁵ is independently hydrogen or C₁₋₆ alkyl,

R⁶ is hydrogen or C₁₋₆ alkyl,

R⁷ is C₁₋₆ alkyl substituted with 1-4 hydroxyl groups,

a is 0-3, and

b is 0-1.

In a specific embodiment, the ketocarboxy ester is of the formula (IIa):

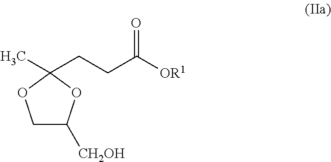

wherein R¹ is a C₈₋₃₆ alkyl, specifically a C₁₂₋₁₇ alkyl, PPO, PEO, or mixed PPO-PEO. Ketocarboxy ester (Ha) is an ester of the glyceryl ketal of levulinic acid (LGK). LGK stearester is obtained when R¹ is a stearyl (C₁₈) group in formula (IIa), and LGK laurester is obtained when R¹ is a lauryl group (C₁₂) in formula (IIa).

In another specific embodiment, the ketocarboxy ester is of the formula (IIb):

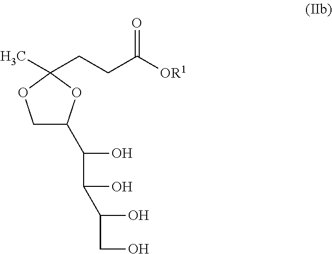

wherein R¹ is a C₈₋₃₆ alkyl, specifically a C₁₂₋₁₇ alkyl, or an alkylene oxide of the formula (CₙH₂ₙO)CₙH₂ₙOR^{a} wherein n is 1-3, m is 1-40, 1 to 30, or 2-20 and R^{a} is H or CₙH₂ₙ₊₁ wherein n is 1 to 3, specifically PPO, PEO, or mixed PPO-PEO.

The ketal adduct can also be a ketocarboxy ester of the formula (III):

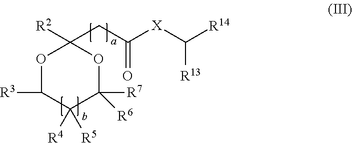

wherein

X is O or NR^{b} wherein R^{b} is hydrogen or an unsubstituted, saturated, or unsaturated C₁₋₃₆ alkyl,

R² is hydrogen or C₁₋₃ alkyl,

each R³, R⁴, and R⁵ is independently hydrogen or C₁₋₆ alkyl,

R⁶ is hydrogen or C₁₋₆ alkyl,

R⁷ is C₁₋₆ alkyl substituted with 1-4 hydroxyl groups,

R¹³ is C₅₋₃₀ alkyl substituted with 1-4 hydroxyl groups,

R¹⁴ is —R¹⁵C(═O)OR¹⁶, wherein R¹⁵ and R¹⁶ are C₁₋₂₀ alkyl,

a is 0-3, and

b is 0-1.

In a specified embodiment, the ketocarboxy ester is of the formula (IIIa) or (IIIb):

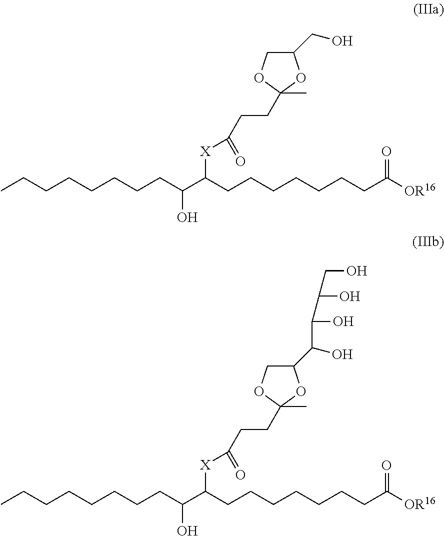

wherein X and R¹⁶ are as defined above in formula (III).

The ketal adduct can be a bisketal adduct of the formula (IV):

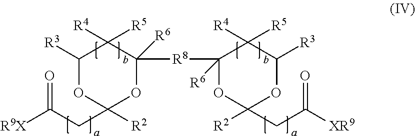

wherein

each X is independently O or NR^{b} wherein R^{b} is hydrogen or an unsubstituted, saturated, or unsaturated C₁₋₃₆ alkyl,

R² is hydrogen or C₁₋₃ alkyl,

each R³, R⁴, and R⁵ is independently hydrogen or C₁₋₆ alkyl,

R⁶ is hydrogen or C₁₋₆ alkyl,

R⁸ is —CR¹⁰— or —CR¹¹CR¹²— wherein R¹⁰, R¹¹ and R¹² are independently hydrogen, hydroxy, or an oxyalkylene of the formula (OCₙH₂ₙ)ₚOR^{a} wherein n is 1-3, p is 1-1000, 2-500, or 2-100, or 2-50, or 2-30, and R^{a} is H or CₙH₂ₙ₊₁ wherein n is 1 to 3,

R⁹ is C₁₋₂₀ alkyl,

a is 0-3, and

b is 0-1.

In specific embodiments, the bisketal adducts have the formula (IVa), (IVb), (IVc), or (IVd):

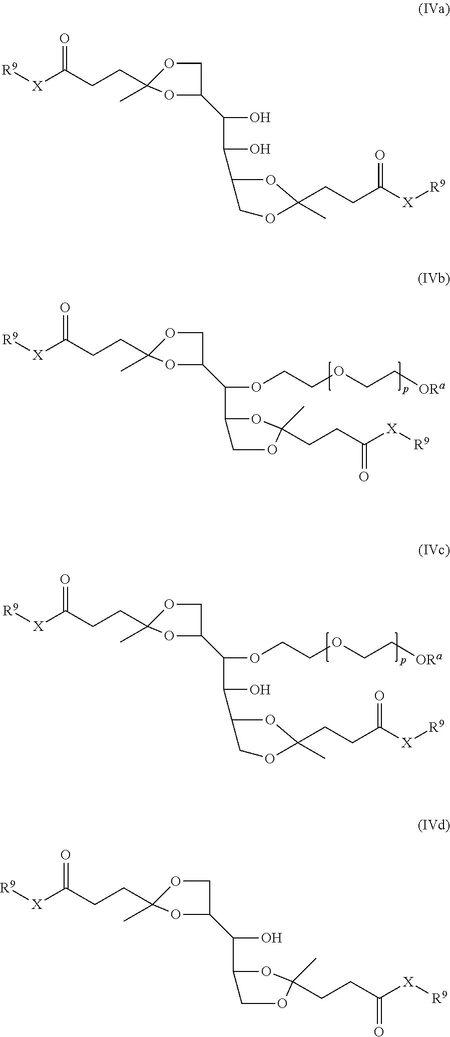

wherein X is O or NR^{b} wherein R^{b} is hydrogen or an unsubstituted, saturated, or unsaturated C₁₋₃₆ alkyl, p is 1-1000, specifically 2-500, 2-100, 2-50, or 2-30, and R⁹ is a C₁₋₆ alkyl group. In an embodiment, X is O. In another embodiment, X is NR^{b}.

The ketal adduct can also have a structure (V):

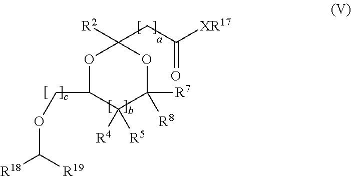

wherein

each X is independently O or NR^{b} wherein R^{b} is hydrogen or an unsubstituted, saturated, or unsaturated C₁₋₃₆ alkyl,

R² is hydrogen or C₁₋₃ alkyl,

each R⁴, R⁵, R⁷, and R⁸ is independently hydrogen or C₁₋₆ alkyl,

R¹⁷ is substituted or unsubstituted, saturated or unsaturated C₁₋₃₆ alkyl,

R¹⁸ is C₆₋₃₀ alkyl,

R¹⁹ is —R¹⁵C(═O)OR¹⁶, wherein R¹⁵ and R¹⁶ are C₁₋₂₀ alkyl,

each a and c is independently 0-3, and

b is 0-1.

In specific embodiments, the ketal adduct is of the formula (Va):

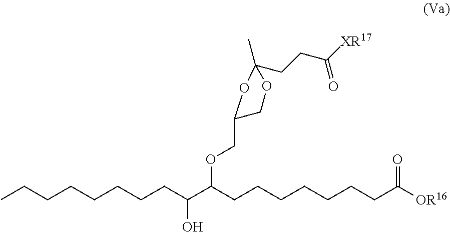

wherein X, R¹⁶, and R¹⁷ are as defined above in formula (V).

Ketal adducts can also have a structure of formula (VI):

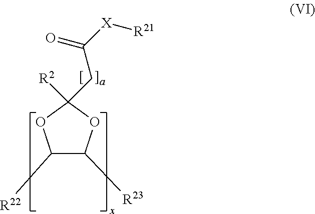

wherein

each X is independently O or NR^{b} wherein R^{b} is hydrogen or an unsubstituted, saturated, or unsaturated C₁₋₃₆ alkyl,

R² is hydrogen or C₁₋₃ alkyl,

R²¹ is substituted or unsubstituted C₁₋₃₆ alkyl,

R²³ is substituted or unsubstituted C₈₋₃₆ alkyl,

R²³ is —R¹⁵C(═O)OR¹⁶, wherein R¹⁵ and R¹⁶ are C₁₋₂₀ alkyl,

a is 0-3, and

x is 1-10.

In a specific embodiment, the ketal adducts of formula (VI) has a structure represented by formula (VIa):

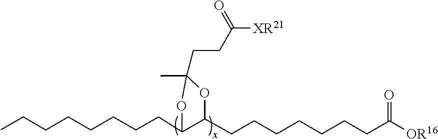

wherein X, R¹⁶, R²¹, and x are as defined above in Formula (VI).

Ketal surfactants of formula (I) can be obtained by methods known in the art, for example by reacting a long-chain linear aliphatic primary or secondary amines of formula (VII) with ketocarboxy esters of formula (VIII).

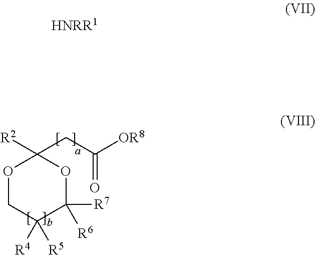

wherein each of R, R₁, R₂, R₃, R₄, R₅, R₆, R₇, a, and b are as defined above in formula I and R⁸ is hydrogen, a C₁₋₄ alkyl group, specifically a methyl or ethyl group, or other activating group for displacement by the amine (VII).

The reaction between amine (VII) and ketocarboxy ester (VIII) can be performed either with or without a catalyst, for example an alkoxide, tertiary amine, or other catalyst. When a catalyst is desired to increase the reaction kinetics, the present application is not limited to a specific catalyst or an amount of catalyst. One of ordinary skill in the art can practice many variations on the part of the catalyst composition and the amounts used in the preparation described herein.

Elevated temperatures can be used to accelerate the reaction, particularly with when no catalyst or a less reactive catalyst is present; however, the temperature of the reaction mixture is not critical for succeeding in making a quantity of the product, as even with less active catalysts the reaction still proceeds to yield the desired compounds. It is preferred, however, that low-cost catalysts that impart minimal or negligible corrosion effects on equipment be used in the synthesis, and that have low volatility, toxicity, and environmental impacts, or that can be easily neutralized to innocuous compounds.

The reaction can advantageously be conducted with concomitant removal of side products, for example R⁸OH when R⁸ is a C₁₋₄ alkyl group.

Ketocarboxy esters (VIII) can be obtained using the procedures described in WO 09/032905, for example, which describes the synthesis of the alkyl ester of an adduct of levulinic acid and glycerol of formula (VIIIa)

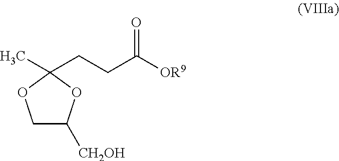

wherein R⁹ is an alkyl group, for example a C₁₋₆ alkyl group. When R⁹ in formula VIIIa is ethyl, the compound is the reaction product of ethyl levulinate with glycerine, i.e., 1,3-dioxolane-2-propanoic acid, 4-(hydroxymethyl)-2-methyl-, ethyl ester (DPHME). Many of the compounds falling within the scope of formula (VIII) can be bio-sourced. For example, levulinic acid can be produced by the thermochemical treatment of various carbohydrates such as cellulose; subsequent esterification with bio-sourced alkanols and ketalization of the levulinate ester with polyhydroxy compounds such glycerol or propylene glycol produces a bioderived compound for further reaction with amine (VII).

It has been found that the ketal surfactants, find use as emulsifiers in emulsions, particularly in oil/water emulsions. Without being bound to theory, it is believed that the ketal amide is amphiphilic, wherein the polar moiety is comprised of the terminal hydroxyl group, the two endocyclic oxygens, and the amide, with the nonpolar moiety being the linear hydrocarbon. However, it is to be understood that the ketal surfactants can have more than one function in a specific composition, including one or more of solubilization, solvent coupling, surface tension reduction, and the like. In a highly advantageous feature, selection of the specific substituents, and a and b in the ketal surfactants allows the chemical and physical properties of the ketal surfactants to be adjusted to achieve desired properties, for example cleaning (as a detergent) or emulsification in a variety of emulsions with different dispersed and continuous phases. For example, surfactants based on structure A, which have a single hydrophobic “tail,” emulsify oils in water but may not achieve the desired HLB (hydrophilic-lipophilic balance) range for a detergent. Typically, for detergents, a HLB range of 10 to 20 is desirable.

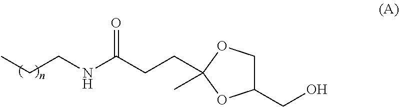

Surfactants based on structures (B) and (C) on the other hand, have two tails. Without wishing to be bound by theory, it is believed that twin tail surfactants play a more active role in the delivery than just as an emulsifier. Accordingly, these surfactants may provide improved surface activating and cleaning properties than the single tailed surfactants based on structure A.

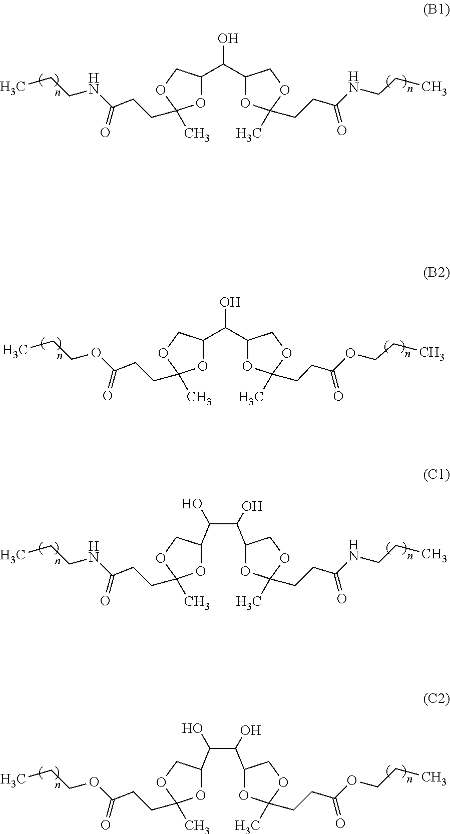

The hydroxyl group in the surfactants of structures (B) and (C) can further react with polyethylene glycol to provide the surfactants (D1)-(D5) below.

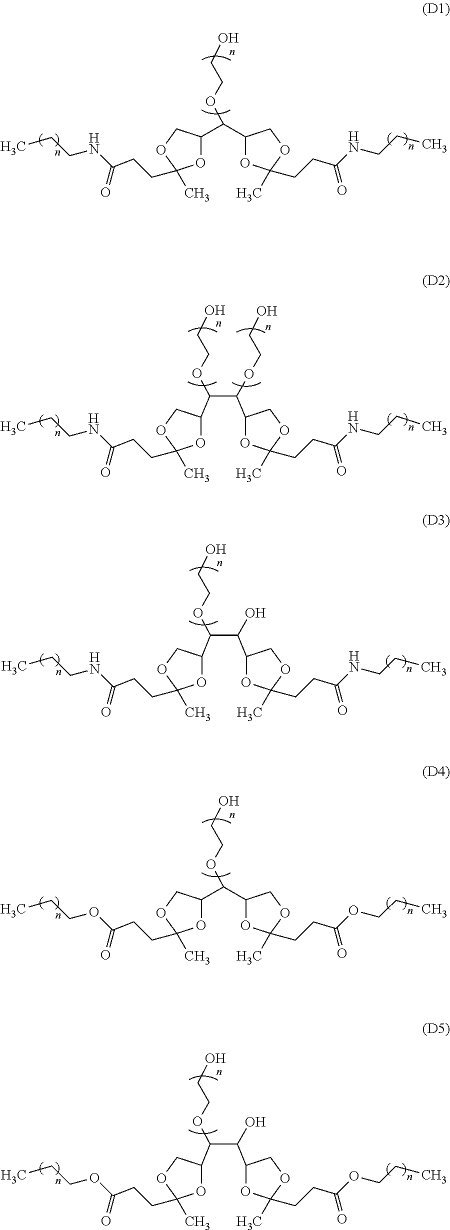

In the foregoing structures, each n in the amide or ester group is independently 2 to 35, and each n in the polyoxyethylene is 2-1000, 2-500, or 2-100, or 2-50, or 2-30. The value of each n can, of course, be varied in order to provide the desired surfactant/emulsification characteristics.

Thus, in an embodiment, an emulsion comprises a dispersed liquid phase, a dispersed continuous phase, and a ketal surfactant. The dispersed phase can be hydrophobic (e.g., an organic liquid such as an oil) or hydrophilic (e.g., an aqueous system or water), that is, the emulsion can be an O/W or W/O emulsion. The ketal amide can be used in multiple emulsions, for example in water-in-oil-in-water (W/O/W) emulsions, oil-in-water-in-oil (O/W/O), and the like. In an embodiment, the emulsion is an O/W emulsion. When the emulsion is an O/W emulsion, R⁷ in ketal amide (I) can be a C₁₋₃ alkyl substituted with a hydroxyl group, specifically a ketal amide of formula (Ia).

The ketal surfactant is present in an amount effective to perform emulsification. Such amounts can vary depending on the specific ketal amide used and the types and relative amounts of dispersed and continuous phases, and can be readily determined by one of skill in the art without undue experimentation. For example, the ketal amide can be present in an amount of about 0.1 to about 10 wt. %, more specifically about 0.5 to about 8 wt. %, and still more specifically about 1 to about 7 wt. %, each based on the total weight of the emulsion.

The relative amounts and identity of the dispersed and continuous phases in the emulsions depends on the particular application, and can vary widely. For example, the emulsion can be a W/O emulsion where the continuous phase is hydrophobic and is present in an amount of about 51 wt. % to about 99 wt. % of the emulsion. In another embodiment, the emulsion is an O/W emulsion where the continuous phase is aqueous and is present in amount of about 51 wt. % to about 99 wt. % of the emulsion. However, the emulsions are not limited to these exemplary embodiments.

A wide variety of liquids can be used in the emulsions, and are selected based on the particular application and properties desired, provided that at least two liquids are used that are immiscible in the presence of the other components of the emulsion. A first liquid is generally a hydrophobic liquid, for example an organic liquid such as an oil. The oils used can be natural or synthetic oils such as vegetable or silicone oils, where non limiting examples include sesame oil, vegetable oil, peanut oil, canola oil, olive oil, soybean oil, black truffle oil, oil derived from seeds such as sunflower seeds, grape seeds, or flax seeds, oil derived from nuts such as macadamia nuts or pine nuts crude oil or motor oil, including single-grade oils such as mineral oil or multi-grade oil, diesel oil, mineral oil, hydrogenated and unhydrogenated olefins including polyalpha-olefins, linear and branched olefins, and the like, fluorocarbons, including perfluorinated compounds, poyldiorganosiloxanes, and esters of fatty acids, specifically straight chain, branched and cyclic alkyl esters of fatty acids. The oil phase can further be a combination of one or more different oils.

A second liquid is generally a hydrophilic liquid, for example a water-soluble organic liquid or water. Exemplary hydrophilic liquids include, but are not limited to polyhydric alcohols (polyols) or water. Examples of polyhydric alcohols include polyalkylene glycols and alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin, and combinations comprising at least one of the foregoing.

Other components may be present in the emulsions depending on the intended use of the emulsions. For example, emulsions intended for personal care compositions can further comprise an active agent (e.g., a medicament or sunscreen), fragrance, pigment, dye, cosolvent, pH adjusting agent, preservatives, and the like.

Methods for forming emulsions using the ketal surfactants are known in the art, and generally include combining the components to form the continuous and discontinuous phases and the ketal amide, with agitation to disperse the component forming the discontinuous phase in the continuous phase. The combining and agitation may be conducted in any order; in an embodiment, the component forming the discontinuous phase is added to the component forming the continuous phase with agitation. Agitation can be by any means, for example, by hand stirring, aeration, propeller agitation, turbine agitation, colloid milling, homogenizing, high-frequency or ultrasonic oscillation (sonication), micro-fluidization and the like. In an embodiment, a homogenizer is used. The emulsion may be further processed, for example to reduce the size of the droplets of the dispersed phase. The processing step may be conducted by homogenization or other suitable methods known to those skilled in the art. Any components used in the emulsion other than the at least two immiscible liquids and the ketal surfactant can be present either initially before emulsification, or added separately or in combination with any other component.

The emulsions thus formed can be characterized by a large particle size (typically greater than 300 nanometers), a smaller particle size, e.g., from 300 to 140 nanometers, or even a particle size of less than 140 nanometers. Use of the ketal surfactants can result in stable emulsions, for example emulsions that are stable when stored at room temperature for up to one week, one month, or one year.

It will be understood that the present application also encompasses the use of an emulsifier as described above for stabilizing emulsions, as well as in other products, such as drug delivery, food, and cosmetic products, biocide compositions, including agrochemical and residential/municipal pesticides, herbicides, rodenticides and fungicides, comprising an emulsion or having the form of an emulsion, wherein the above-described ketal amide is present as an emulsifier. In some products the emulsion is formed during use of the product, for example certain cleaning products. Thus, the ketal amide of formula (I), specifically of formula (Ia), can be used in many applications, particularly in the drug delivery, food, cleaning, fire extinguishing media, and personal care applications. Some non-limiting examples of uses for the ketal amide of formula (I), specifically of formula (Ia), are in personal care products, for instance in hair conditioners, shampoos, emollients, lotions, and creams; as replacements for protein-based emulsifiers such as casein or caseinates, or other emulsifiers, such as glycerol monostearate or glycerol distearate, or to replace eggs in bakery products or in emulsified sauces; as complexes to be used to create an elastic, gelled foam, e.g., as foam booster in for example whipped creams, meringues, shampoos, shaving creams, bath or shower gels, and liquid soaps; or as complexes used in papermaking.

The ketal amide of formula (I), specifically formula (Ia), can also be used in compositions containing other emulsifiers or surfactants. The ketal amide can work with other emulsifiers to stabilize an emulsion, or the other surfactants can perform different functions, such as soil removal or foaming action. Surfactants for various uses are known in the art, for example various anionic surfactants can be present for cleaning, emulsion stabilization, foaming, and the like. Cationic surfactants can be present for hair conditioning or skin conditioning. Nonionic surfactants can be present for emulsification or delivery of agents (fragrances, actives, and the like.) It is again to be stated that these embodiments are non-limiting examples as to the uses of the ketal surfactants. The ketal surfactants can also be used in compositions where a surfactant is desired for a variety of applications, including personal care, pharmaceutical, agricultural, and cleaning, for example household or commercial cleaning or for cleaning oil spills.

In other embodiments, the compositions of the invention can include at least one of the ketal surfactants and a solvent, such as water or an organic solvent. The compositions can be solutions, emulsions or microemulsions. The compositions can also contain additional components, such as pigments and resins. The compositions can be made into formulations which can be sprayed, poured, spread, coated, dipped or rolled.

Set forth below are some embodiments of the ketal amides, methods for making ketal amides, ketocarboxy esters, ketal adducts, and compositions comprising these ketal compounds.

In an embodiment, a ketal amide has formula (I), wherein R is hydrogen or C₁₋₈ alkyl; R¹ is substituted or unsubstituted, saturated or unsaturated C₁₋₃₆ alkyl, or an alkylene oxide of the formula (CₙH₂ₙO)ₚCₙH₂ₙOR^{a} wherein n is 1-4, p is 1-1000 and IV is H or CₙH₂ₙ₊₁ wherein n is 1 to 4, R² is hydrogen or C₁₋₃ alkyl, each R³, R⁴, and R⁵ is independently hydrogen or C₁₋₆ alkyl, R⁶ is hydrogen or C₁₋₆ alkyl, R⁷ is C₁₋₆ alkyl substituted with 1-4 hydroxyl groups, a is 0-3, and b is 0-1.

In specific embodiments of the foregoing ketal amide, one or more of the following condition can apply: (i) R is hydrogen, R¹ is substituted or unsubstituted, saturated or unsaturated C₈₋₃₆ alkyl, PPO, PEO, or mixed PPO-PEO, R² is methyl, each R³, R⁴, and R⁵ is independently hydrogen or C₁₋₃ alkyl, R⁶ is hydrogen, R⁷ is C₁₋₆ alkyl substituted with 1-2 hydroxyl groups, a is 1-3, and b is 0-1; (ii) R¹ is substituted or unsubstituted, saturated or unsaturated C₈₋₃₆ alkyl, PPO, PEO, or mixed PPO-PEO, R² is methyl, R³ is hydrogen, R⁶ is hydrogen, R⁷ is C₁₋₃ alkyl substituted with 1-2 hydroxyl groups, a is 2-3, and b is 0-1; (iii) the ketal amide has the structure (Ia), wherein R¹ is an unsubstituted, saturated, or unsaturated C₈₋₃₆ alkyl, PPO, PEO, or mixed PPO-PEO; (iv) R¹ is an unsubstituted, saturated, or unsaturated C₁₀₋₂₀ alkyl, PPO, PEO, or mixed PPO-PEO; (v) R¹ is an unsubstituted, saturated, or unsaturated C₁₂₋₁₈ alkyl; and/or (vi) R¹ is an unsubstituted, saturated C₁₂₋₁₈ alkyl, PPO, PEO, or mixed PPO-PEO.

A method of producing the foregoing ketal amide comprises contacting an amine of formula (VII), wherein R is hydrogen or C₁₋₈ alkyl and R¹ is C₁₋₃₆ alkyl, PPO, PEO, or mixed PPO-PEO, with a ketocarboxy esters of formula (VIII), wherein R² is hydrogen or C₁₋₃ alkyl, each R³, R⁴, and R⁵ is independently hydrogen or C₁₋₆ alkyl, R⁶ is hydrogen or C₁₋₆ alkyl, R⁷ is C₁₋₆ alkyl substituted with 1-4 hydroxyl groups, R⁸ is hydrogen or a C₁₋₄ alkyl group, a is 0-3, and b is 0-1, under reaction conditions to produce the ketal amide.

In another embodiment, an emulsion comprises a continuous phase; a discontinuous phase dispersed in the continuous phase; and a ketal amide of any of the foregoing embodiments.

In specific embodiments of the emulsion, one or more of the following conditions can apply: (i) the continuous phase is an aqueous phase or a water phase; (ii) the discontinuous phase is an aqueous phase or a water phase; and/or (iii) the emulsion comprises from 0.1 to 10 weight percent of the ketal amide, based on the total weight of the emulsion.

A method for the manufacture of the emulsion comprises combining a first liquid component for forming a continuous phase, a second liquid component for forming a discontinuous phase, and the ketal amide; and dispersing second component in the first component to produce the emulsion.

In specific embodiments, a person care composition, a drug delivery composition, a cleaning composition, or a biocide composition such as a pesticide comprises the emulsion of any of the foregoing embodiments.

In another embodiment, a composition comprises: an oil; and a ketal amide of any of the foregoing embodiments. The composition can further comprise one or more of: a biocide active agent, a fragrance, or water.

Various ketocarboxy esters are also disclosed. In an embodiment, a ketocarboxy ester has a formula (II), wherein R¹ is substituted or unsubstituted, saturated or unsaturated C₈₋₃₆ alkyl, PPO, PEO, or mixed PPO-PEO, R² is hydrogen or C₁₋₃ alkyl, each R³, R⁴, and R⁵ is independently hydrogen or C₁₋₆ alkyl, R⁶ is hydrogen or C₁₋₆ alkyl, R⁷ is C₁₋₆ alkyl substituted with 1-4 hydroxyl groups, a is 0-3, and b is 0-1. In a specific embodiment, the ketocarboxy ester has the structure (IIa) or (IIb): wherein R¹ is substituted or unsubstituted, saturated or unsaturated C₈₋₃₆ alkyl, PPO, PEO, or mixed PPO-PEO.

In another embodiment, a ketocarboxy ester has formula (III), wherein X is O or NR^{b} wherein R^{b} is hydrogen or an unsubstituted, saturated, or unsaturated C1-36 alkyl, R² is hydrogen or C₁₋₃ alkyl, each R³, R⁴, and R⁵ is independently hydrogen or C₁₋₆ alkyl, R⁶ is hydrogen or C₁₋₆ alkyl, R⁷ is C₁₋₆ alkyl substituted with 1-4 hydroxyl groups, R¹³ is C₅₋₃₀ alkyl substituted with 1-4 hydroxyl groups, R¹⁴ is —R¹⁵C(═O)OR¹⁶, wherein R¹⁵ and R¹⁶ are C₁₋₂₀ alkyl, a is 0-3, and b is 0-1. In specific embodiments, the ketocarboxy ester has the structure (IIIa) or (IIIb), wherein X is O or NR^{b} wherein R^{b} is hydrogen or an unsubstituted, saturated, or unsaturated C₁₋₃₆ alkyl, and R¹⁶ is C₁₋₂₀ alkyl.

In yet another embodiment, a ketal adduct has the formula (IV), wherein each X is independently O or NR^{b} wherein R^{b} is hydrogen or an unsubstituted, saturated, or unsaturated C₁₋₃₆ alkyl, R² is hydrogen or C₁₋₃ alkyl, each R³, R⁴, and R⁵ is independently hydrogen or C₁₋₆ alkyl, R⁶ is hydrogen or C₁₋₆ alkyl, R⁸ is —CR¹⁰— or —CR¹¹CR¹²— or wherein R¹⁰, R¹¹ and R¹² are independently hydrogen hydroxy, or an oxyalkylene of the formula (OCₙH₂ₙ)ₚOR^{a} wherein n is 1-3, p is 1-1000, and R^{a} is H or CₙH₂ₙ₊₁ wherein n is 1 to 3, R⁹ is C₁₋₂₀ alkyl, a is 0-3, and b is 0-1. In specific embodiments, the ketal adduct has the structure (IVa), (IVb), (IVc) or (IVd).

In still another embodiment, a ketal adduct having the formula (V), wherein each X is independently O or NR^{b} wherein R^{b} is hydrogen or an unsubstituted, saturated, or unsaturated C1-36 alkyl, R² is hydrogen or C₁₋₃ alkyl, each R⁴, R⁵, R⁷, and R⁸ is independently hydrogen or C₁₋₆ alkyl, R¹⁷ is substituted or unsubstituted, saturated or unsaturated C₁₋₃₆ alkyl, R¹⁸ is C₆₋₃₀ alkyl, R¹⁹ is —R¹⁵C(═O)OR¹⁶, wherein R¹⁵ and R¹⁶ are C₁₋₂₀ alkyl, each a and c is independently 0-3, and b is 0-1. In specific embodiments, the ketal adduct has the structure (Va), wherein X is O or NR^{b} wherein R^{b} is hydrogen or an unsubstituted, saturated, or unsaturated C₁₋₃₆ alkyl, R¹⁶ is C₁₋₂₀ alkyl, and R¹⁷ is substituted or unsubstituted, saturated or unsaturated C₁₋₃₆ alkyl.

In another embodiment, a ketal adduct has the formula (VI), wherein each X is independently O or NR^{b} wherein R^{b} is hydrogen or an unsubstituted, saturated, or unsaturated C₁₋₃₆ alkyl, R² is hydrogen or C₁₋₃ alkyl, R²¹ is substituted or unsubstituted C₁₋₃₆ alkyl, R²³ is substituted or unsubstituted C₈₋₃₆ alkyl, R²³ is —R¹⁵C(═O)OR¹⁶, wherein R¹⁵ and R¹⁶ are C₁₋₂₀ alkyl, a is 0-3, and x is 1-10. In specific embodiments, the ketal adduct has the structure (VIa), wherein each X is independently O or NR^{b} wherein R^{b} is hydrogen or an unsubstituted, saturated, or unsaturated C₁₋₃₆ alkyl, R¹⁶ is C₁₋₂₀ alkyl, and R²¹ is substituted or unsubstituted C₁₋₃₆ alkyl, x is 1-10.

ketal adducts having the structure (B1), (B2), (C1), (C2), (D1), (D2), (D3), (D4) and (D5) are also disclosed, wherein each n in the ester or amide group is 2 to 35, and each n in the polyoxyethylene is 2-1000.

In an embodiment, a composition comprises at least one ketal amides, ketocarboxy esters, and ketal adduct of any of the foregoing embodiments, and a solvent.

In specific embodiments of the foregoing composition, one or more of the following can apply: (i) the solvent is water; (ii) the solvent is not water; (iii) the composition further comprises water; (iv) the composition comprises at least two ketal compounds of the foregoing embodiments; (v) the composition is an emulsion; (vi) the composition is a microemulsion; (vii) the composition is a solution; (viii) the composition further comprises a pigment; (IX) the composition further comprises a resin; and/or (X) the composition can be sprayed, poured, spread, coated, dipped or rolled.

The following non-limiting examples further illustrate various embodiments of the present application.

### EXAMPLES

Gel permeation chromatography (GPC) was used to determine the monomer conversion as well as the molecular weight of the product.

Differential scanning calorimetry (DSC) was performed over the temperature range of 40-150° C., using a ramp rate of 10° C./minute to determine the melting temperature, Tₘ, the crystallization temperature, T_{c}, and the enthalpy of transition, ΔH, of the final product.

Emulsification is tested in accordance with the procedure described by I. Roland et al., in the International Journal of Pharmacology, Volume 263, pages 85-94 (2003).

The following components were used:

TABLE 1

| Component | Source |
|---|---|
| Octadecylamine | Acros, 90% purity |
| DPHME* | Segetis, Inc. |
| Hexane | Fisher HPLC Grade 99.9% |
| Dodecylamine | Acros Organics 98% |
| Sesame oil | Jeen International Corporation NF/USP |
| Cocoamide DEA | The Chemistry Store.com |
| Rhodoline 643, defoamer | Rhodia, Inc. |

*1,3-Dioxolane-2-propanoic acid, 4-(hydroxymethyl)-2-methyl-, ethyl ester

### Example 1

### Synthesis of 1,3-Dioxolane-2-propanoic acid, 4-(hydroxymethyl)-2-methyl-, stearamide (DPHMS)

1,3-Dioxolane-2-propanoic acid, 4-(hydroxymethyl)-2-methyl-, stearamide was synthesized by adding 80.15 g (0.30 mol) of octadecylamine and 66.18 g (0.30 mol) of DPHME to a 500 mL three-neck flask that was equipped with a mechanical stirrer and a Dean-Stark apparatus. The contents were degassed by three repetitions of evacuating the flask to 1 torr (133 Pa) for 5 minutes and refilling the flask with nitrogen. Under nitrogen overpressure, the reaction mixture was heated to 220° C. for 1.5 hours and 240° C. for 5 hours. The apparatus was reconfigured for nitrogen sweep and heated to 240° C. for 2 hours, at which point collection of volatiles in the Dean-Stark trap had subsided. GPC analysis indicated 97.4% monomer conversion. The crude yield was 128.5 g. Recrystallization from hexane proved effective for removing color and residual DPHME, resulting in 99.3% purity of the final product.

DSC analysis on the final product was performed over the temperature range of −40-150° C. to determine that Tₘ=52.09° C., ΔH=85.9 J/g; T_{c}=45.6° C., and ΔH=89.3 J/g.

### Example 2

### Synthesis of 1,3-Dioxolane-2-propanoic acid, 4-(hydroxymethyl)-2-methyl-, lauramide (DPHML)

1,3-Dioxolane-2-propanoic acid, 4-(hydroxymethyl)-2-methyl-, lauramide was synthesized as follows.

The reaction was carried out by adding 39.7 g (0.21 mol) of dodecylamine and 50.1 g (0.23 mol) of DPHME to a 500 mL three-neck flask that was equipped with a magnetic stirrer and a Dean-Stark apparatus. The contents were degassed by three repetitions of evacuating the flask to 10 torr (1.3 kPa) for 1 minute and refilling the flask with nitrogen. Under nitrogen overpressure, the reaction mixture was heated to 210° C. for 20 hours, at which point the 93% of the theoretical volume of volatiles had been collected. The reactor was cooled to 190° C. and 1 torr (133 Pa) vacuum was applied for 45 minutes, leading to collection of an additional 8.3 mL of volatiles. The final product was isolated.

GPC analysis indicated 86% purity of a product whose molecular mass at the peak, Mp, was 506 g/mol and 14% of an oligomer product with an Mp of 898 g/mol.

### Examples 3-6 and Comparative Examples C3-C5

Stability of a sesame oil/water emulsions in the presence of DPHML and cocoamide DEA was tested as a function of freeze/thaw cycles.

Sesame oil/water emulsions were prepared using a 1:1 weight ratio of sesame oil to water in the presence of 3 weight % DPHML (Examples 3-6) or cocoamide diethanolamine (DEA) (Comparative Examples 3-5). 20 microliters of Rhodoline 643 was further added to Example 6.

Examples 3-6 and Comparative Examples C7-9 then underwent the same successive freeze/thaw cycles wherein the samples were placed in a freezer at −20° C. overnight and subsequently thawed. The percent creaming height with freeze thaw cycles is shown in Table 2.

TABLE 2

| | | | | | Avg of | | | | Avg of |
|---|---|---|---|---|---|---|---|---|---|
| Cycle | 3 | 4 | 5 | 6 | 3-6 | C3 | C4 | C5 | C3-C5 |
| No. | Creaming Height (%) | | | | | | | | |
| 1 | 92 | 80 | 91 | 93 | 89 | 100 | 96 | 97 | 97 |
| 2 | 92 | 64 | 84 | 92 | 83 | 75 | 93 | 91 | 86 |
| 3 | 88 | 62 | 79 | 86 | 79 | 78 | 85 | 86 | 83 |
| 4 | 79 | 61 | 76 | 79 | 74 | 75 | 78 | 73 | 75 |
| 5 | 75 | 61 | 67 | 78 | 70 | 72 | 68 | 62 | 67 |

*Example 6 also contains approximately 20 microliters of the defoamer Rhodoline 643.

The data in Table 2 shows that the percent creaming height decreases with the number of freeze/thaw cycles.

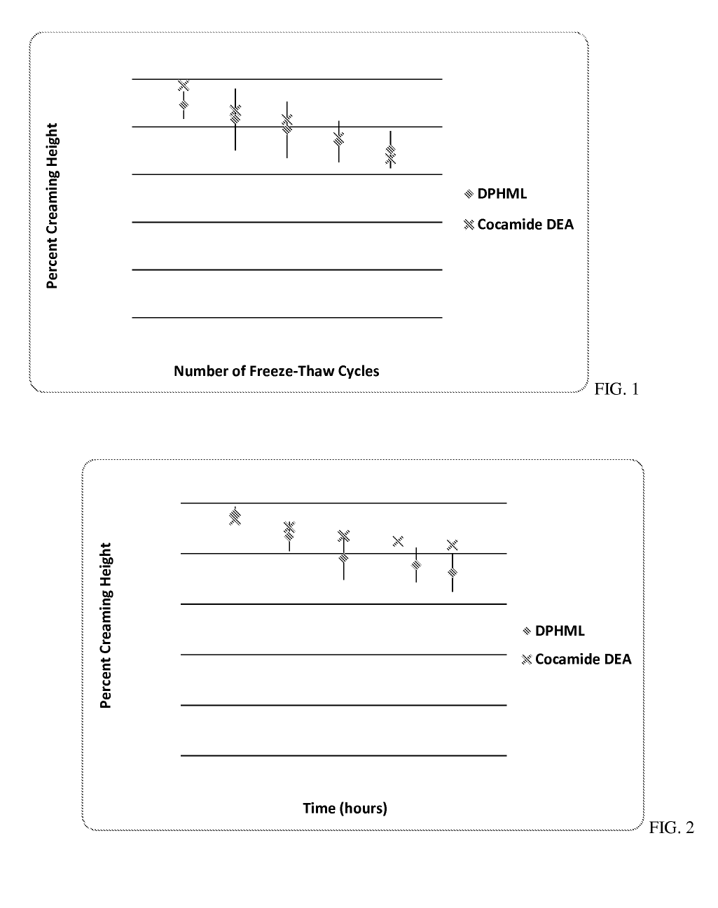

The average percent creaming height as a function of freeze thaw cycles from Examples 3-6 and Comparative Examples C3-05 are plotted in FIG. 1. It can be seen from the data that by the fifth cycle, the percent creaming height of the emulsions is greater when DPHML lauramide is used as the emulsifier, indicating that DPHML provides greater stability than cocoamide DEA.

### Examples 7-9 and Comparative Examples C7-C9

Stability of sesame oil/water emulsions in the presence of DPHML and cocoamide DEA was tested as a function of time at 35° C.

The samples prepared in Examples 3-5 were re-emulsified and are Examples 7-9, respectively. Sesame oil/water emulsions for Comparative Examples C7-C9 were prepared as in Comparative Examples 3-5. Once the samples were stable and at room temperature, they were placed in a forced air oven at 35° C. and the percent creaming height was recorded with time as seen in Table 3.

TABLE 3

| | | | | Avg of | | | | | Avg of |
|---|---|---|---|---|---|---|---|---|---|
| Time | 7 | 8 | 9 | 7-9 | Time | C7 | C8 | C9 | C7-C9 |
| (hours) | Creaming Height (%) | | | | (hours) | Creaming Height (%) | | | |
| 1 | 99 | 100 | 93 | 97 | 1 | 94 | 93 | 94 | 94 |
| 2 | 93 | 86 | 81 | 87 | 2 | 92 | 89 | 89 | 90 |
| 3 | 87 | 78 | 70 | 78 | 3 | 89 | 85 | 86 | 87 |
| 4.3 | 82 | 76 | 69 | 76 | 4 | 86 | 84 | 84 | 85 |
| 5 | 80 | 72 | 65 | 72 | 5 | 85 | 81 | 83 | 83 |

The data in Table 4 shows that the percent creaming height decreases with the time.

The average percent creaming height as a function of time from Examples 7-9 and Comparative Examples 7-9 is plotted in FIG. 2.

### Examples 10-12 and Comparative Examples C10-C12

Stability of sesame oil/water emulsions in the presence of DPHML and cocoamide DEA were tested as a function of time at 20° C.

Examples 10-12 containing 1,3-dioxolane-2-propanoic acid, 4-(hydroxymethyl)-2-methyl-, ethyl ester lauramide and Comparative Examples C10-C12 containing cocoamide DEA were prepared according to the procedure of Examples 3-5. Once the samples were stable they were kept at room temperature, 20° C., and the percent creaming height was recorded with time as shown in Table 4.

TABLE 4

| | | | | Avg of | | | | | Avg of |
|---|---|---|---|---|---|---|---|---|---|
| Time | 10 | 11 | 12 | 10-12 | Time | C10 | C11 | C12 | C10-C12 |
| (hours) | Creaming Height (%) | | | | (hours) | Creaming Height (%) | | | |
| 1.5 | 100 | 100 | 100 | 100 | 1.1 | 96 | 95 | 95 | 96 |
| 2.4 | 100 | 100 | 100 | 100 | 2.1 | 94 | 91 | 92 | 92 |
| 3.4 | 100 | 100 | 100 | 100 | 3.5 | 93 | 90 | 90 | 91 |
| 4.3 | 100 | 100 | 100 | 100 | 4.8 | 88 | 84 | 86 | 86 |
| 6 | 100 | 100 | 100 | 100 | 5.8 | 87 | 82 | 83 | 84 |

The data in Table 4 shows that the percent creaming height surprisingly does not decrease with time in Examples 10-12, and the emulsion is otherwise stable. The data in Table 7 shows that the percent creaming height decreases with the time in Comparative Examples C10-C12.

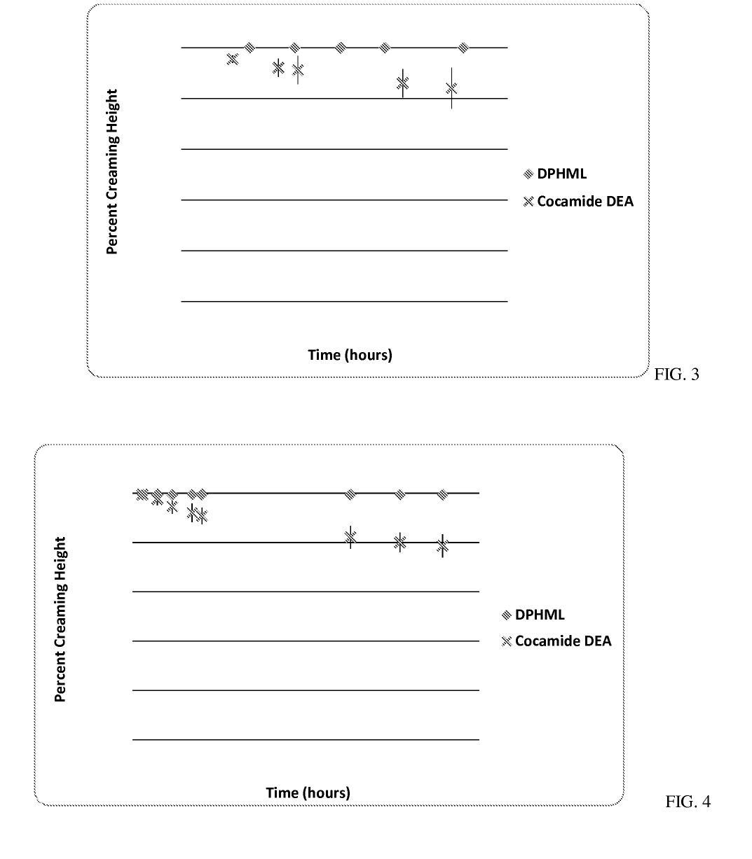

The average percent creaming height as a function of time from Examples 10-12 and Comparative Examples C10-C12 is plotted in FIG. 3. It can be seen from the data that DPHML is a far superior emulsifier at 20° C. compared to cocoamide DEA, as the percent creaming height does not decrease with the DPHML emulsifier.

### Examples 13-15 and Comparative Examples 13-15

Stability of sesame oil/water emulsions in the presence of DPHML lauramide or cocoamide DEA was tested as a function of time at 5° C.

Examples 13-15 were prepared according to the procedure of Examples 3-5. Once the samples were stable they were placed under refrigeration at 5° C., and the percent creaming height was recorded with time as seen in Table 5 (data for cocoamide DEA not shown in Table 5).

TABLE 5

| | | | | |
|---|---|---|---|---|
| Time | 13 | 14 | 15 | Avg of 13-15 |

| | |
|---|---|
| (hours) | Creaming Height (%) |

| | | | | |
|---|---|---|---|---|
| 1 | 94 | 96 | 97 | 96 |
| 2.5 | 94 | 97 | 98 | 96 |
| 4 | 92 | 97 | 97 | 95 |
| 6 | 92 | 97 | 98 | 96 |
| 7 | 93 | 97 | 98 | 96 |
| 22 | 93 | 96 | 98 | 96 |
| 27 | 92 | 98 | 98 | 96 |
| 31.3 | 92 | 97 | 97 | 95 |

The data in Table 5 shows that the percent creaming height in Examples 13-15 surprisingly decreases very little with time and the emulsion is otherwise stable.

The average percent creaming height as a function of time from Examples 13-15 and Comparative Examples C13-C15 is plotted in FIG. 4. It can be seen from the data that DPHML is a far superior emulsifier at 5° C. as compared to cocoamide DEA as the percent creaming height does not decrease with the DPHML emulsifier.

The terminology used herein is for the purpose of describing particular embodiments only and is not intended to be limiting. “Or” means “and/or”. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The endpoints of all ranges directed to the same component or property are inclusive of the endpoint and independently combinable.

Unless otherwise defined, all terms (including technical and scientific terms) used herein have the same meaning as commonly understood by one of ordinary skill in the art to which this invention belongs.

The compounds made by the above-described methods have, in embodiments, one or more isomers. Where an isomer can exist, it should be understood that the invention embodies methods that form any isomer thereof, including any stereoisomer, any conformational isomer, and any cis, trans isomer; isolated isomers thereof; and mixtures thereof.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group. Saturated and unsaturated alkyl, groups can be straight-chained or branched. Unsaturated alkyl groups can have 1, 2, 3, or 4 sites of unsaturation (i.e., one or more double bonds, one or more triple bonds, or a combination thereof) located internally or at a terminal end of the group.

As used herein, a substituted group is a group substituted with one or more (e.g., 1, 2, 3, or 4) substituents independently selected from a C₁ to C₁₀ alkoxy group, a nitro group, a cyano group, a halogen, a C₃ to C₁₀ cycloalkyl group, a C₃ to C₁₀ cycloalkenyl group, a C₃ to C₁₀ cycloalkynyl group, a C₂ to C₁₀ heterocycloalkyl group, a C₂ to C₁₀ heterocycloalkenyl group, a C₂ to C₁₀ heterocycloalkynyl group, a C₆ to C₂₀ aryl group, and a C₂ to C₂₀ heteroaryl group, provided that the substituted atom's normal valence is not exceeded. The prefix “hetero” means that one or more (e.g., 1, 2, or 3) carbon atoms of the group is replaced with S, N, P, O, or Si.

The term “ketal ester” means the cyclic ketal or acetal of a keto acid, semialdehyde, or ester thereof.

All cited patents, patent applications, and other references are incorporated herein by reference in their entirety.

The various embodiments described above are provided by way of illustration only and should not be construed to limit the claims attached hereto. The present invention can suitably comprise, consist of, or consist essentially of, any of the disclosed or recited elements. Thus, the invention illustratively disclosed herein can be suitably practiced in the absence of any element, which is not specifically disclosed herein. Various modifications and changes will be recognized that can be made without following the example embodiments and applications illustrated and described herein, and without departing from the true spirit and scope of the following claims.

What is claimed is:

1. A ketal amide of formula (I): 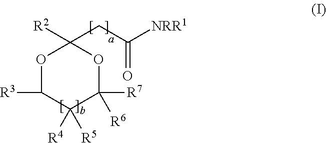
wherein
R is hydrogen or C₁₋₈ alkyl;
R¹ is substituted or unsubstituted, saturated or unsaturated C₁₋₃₆ alkyl, or an alkylene oxide of the formula (CₙH₂ₙO)ₚCₙH₂ₙOR^{a} wherein n is 1-4, p is 1-1000 and R^{a} is H or CₙH₂ₙ₊₁ wherein n is 1 to 4,
R² is hydrogen or C₁₋₃ alkyl,
each R³, R⁴, and R⁵ is independently hydrogen or C₁₋₆ alkyl,
R⁶ is hydrogen or C₁₋₆ alkyl,
R⁷ is C₁₋₆ alkyl substituted with 1-4 hydroxyl groups,
a is 0-3, and
b is 0-1.

2. The ketal amide of claim 1, wherein R is hydrogen, R¹ is substituted or unsubstituted, saturated or unsaturated C₈₋₃₆ alkyl, PPO, PEO, or mixed PPO-PEO, R² is methyl, each R³, R⁴, and R⁵ is independently hydrogen or C₁₋₃ alkyl, R⁶ is hydrogen, R⁷ is C₁₋₆ alkyl substituted with 1-2 hydroxyl groups, a is 1-3, and b is 0-1.

3. (canceled)

4. The ketal amide of claim 1, having the structure (Ia): 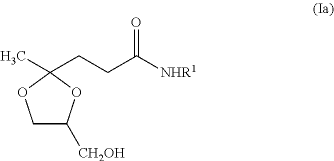
wherein R¹ is an unsubstituted, saturated, or unsaturated C₈₋₃₆ alkyl, PPO, PEO, or mixed PPO-PEO.

5. The ketal amide of claim 1, wherein R¹ is an unsubstituted, saturated, or unsaturated C₁₀₋₂₀ alkyl, PPO, PEO, or mixed PPO-PEO.

6. The ketal amide of claim 1, wherein R¹ is an unsubstituted, saturated, or unsaturated C₁₂₋₁₈ alkyl.

7. The ketal amide of claim 1, wherein R¹ is an unsubstituted, saturated C₁₂₋₁₈ alkyl, PPO, PEO, or mixed PPO-PEO.

8. (canceled)

9. An emulsion comprising:
a continuous phase;
a discontinuous phase dispersed in the continuous phase; and
a ketal amide of claim 1.

10. The emulsion of claim 9, wherein continuous phase is an aqueous phase or a water phase.

11. The emulsion of claim 9, wherein the discontinuous phase is an aqueous phase or a water phase.

12. The emulsion of claim 9, comprising from 0.1 to 10 weight percent of the ketal amide, based on the total weight of the emulsion.

13. (canceled)

14. A composition comprising the emulsion of claim 9, where the composition is a personal care composition, a drug delivery composition, a cleaning composition, or a biocide composition.

15. (canceled)

16. (canceled)

17. (canceled)

18. (canceled)

19. A composition comprising:
an oil; and
a ketal amide of claim 1.

20. The composition of claim 19, further comprising a biocide active agent, a fragrance, water, or a combination comprising at least one of the foregoing.

21. (canceled)

22. (canceled)

23. A ketocarboxy ester of formula (II): 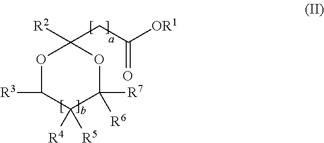
wherein
R¹ is substituted or unsubstituted, saturated or unsaturated C₈₋₃₆ alkyl, PPO, PEO, or mixed PPO-PEO,
R² is hydrogen or C₁₋₃ alkyl,
each R³, R⁴, and R⁵ is independently hydrogen or C₁₋₆ alkyl,
R⁶ is hydrogen or C₁₋₆ alkyl,
R⁷ is C₁₋₆ alkyl substituted with 1-4 hydroxyl groups,
a is 0-3, and
b is 0-1.

24. The ketocarboxy ester of claim 23, having the structure (IIa) or (IIb): 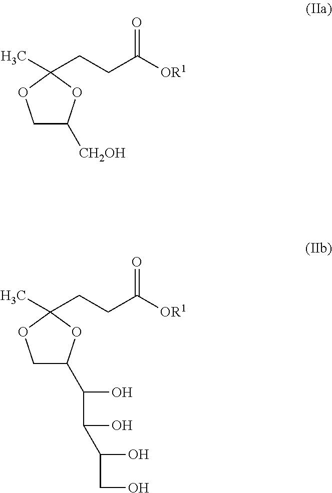
wherein R¹ is substituted or unsubstituted, saturated or unsaturated C₈₋₃₆ alkyl, PPO, PEO, or mixed PPO-PEO.

25. A ketocarboxy ester of the formula (III): 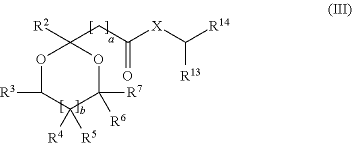
wherein
X is O or NR^{b} wherein R^{b} is hydrogen or an unsubstituted, saturated, or unsaturated C1-36 alkyl,
R² is hydrogen or C₁₋₃ alkyl,
each R³, R⁴, and R⁵ is independently hydrogen or C₁₋₆ alkyl,
R⁶ is hydrogen or C₁₋₆ alkyl,
R⁷ is C₁₋₆ alkyl substituted with 1-4 hydroxyl groups,
R¹³ is C₅₋₃₀ alkyl substituted with 1-4 hydroxyl groups,
R¹⁴ is R¹⁵C(O)OR¹⁶, wherein R¹⁵ and R¹⁶ are C₁₋₂₀ alkyl,
a is 0-3, and
b is 0-1.

26. The ketocarboxy ester of claim 25, having the structure (IIIa) or (IIIb): 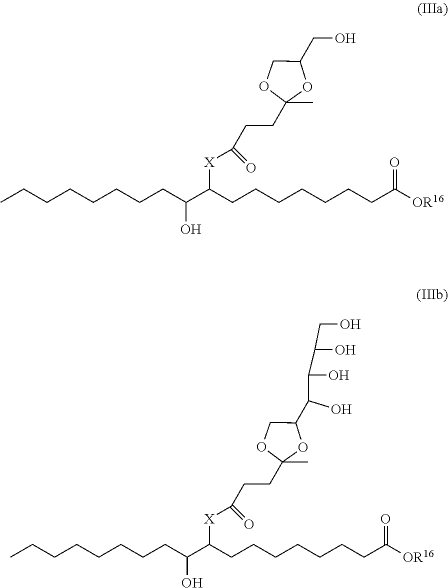
wherein X is O or NR^{b} wherein R^{b} is hydrogen or an unsubstituted, saturated, or unsaturated C₁₋₃₆ alkyl, and R¹⁶ is C₁₋₂₀ alkyl.

27. (canceled)

28. (canceled)

29. (canceled)

30. (canceled)

31. (canceled)

32. (canceled)

33. (canceled)

34. (canceled)

35. A composition comprising at least one ketal of claim 1 and a solvent.

36. (canceled)

37. (canceled)

38. (canceled)

39. (canceled)

40. The composition of claim 35, wherein the composition is an emulsion, microemulsion, or solution.

41. (canceled)

42. (canceled)

43. The composition of claim 35, further comprising a pigment or a resin.

44. (canceled)

45. (canceled)
